(19) United States
(12) Patent Application Publication
(10) Pub. No.: US 2026/0223795 A1
(43) Pub. Date: Aug. 6, 2026

(54) CARRIERS FOR GROWING ALGAE

(71) Applicant: ALGAFILM TECHNOLOGIES LTD, Ancaster (CA)

(72) Inventors: Pierre Lucien COTE, Ancaster (CA); Steven Kristian PEDERSEN, Nottawa (CA)

(21) Appl. No.: 19/147,981

(22) PCT Filed: Jan. 5, 2024
(86) PCT No.: PCT/CA2024/050008

§ 371 Date: Jul. 14, 2025

Related U.S. Application Data
(60) Provisional application No. 63/439,660, filed on Jan. 18, 2023.

(51) Int. Cl. A01G 33/00; C12M 1/00; C12M 1/12; C12M 3/00

(52) U.S. Cl. CPC A01G 33/00; C12M 21/02; C12M 23/04; C12M 23/26; C12M 23/44; C12M 25/06; C12M 29/06

(57) ABSTRACT

Microalgae are grown in a biofilm on carriers. In some examples, the carriers are surfaces with a small angle to the vertical. Optionally, the carriers may have surfaces on both sides of a vertical plane (e.g., an inverted V) or surrounding a vertical axis (e.g., a cone). In some examples, the carriers include two or more sheets of a porous material such as a fabric, screen or mesh. In some examples a surface of the carrier is flexible. In some examples, the carriers provide a specific surface area for algae growth greater than 7 m²/m².

### RELATED APPLICATION

This application claims the benefit of, and priority from, U.S. Provisional Application No. 63/439,660, filed on Jan. 18, 2023, which is incorporated by reference.

### FIELD

This specification relates to carriers, systems and methods useful for growing algae.

### BACKGROUND

Microalgae are photosynthetic organisms from multiple natural groups having a size up to a few hundred micrometers. Their elemental composition is represented by the Redfield ratio: C₁₀₆H₂₆₃O₁₁₀N₁₆P₁. Algae use inorganic carbon through photosynthesis as a source of carbon, and produce oxygen. On a mass basis, every kg of algae produced consumes about 2.0 kg of CO₂. Algae also need nutrients, for example nitrogen and phosphorus, which they extract from water in the form of ammonia, nitrate, or orthophosphate. Every kg of algae produced requires approximately 100 g of inorganic nitrogen and 10 g of orthophosphate (measured as phosphate).

Species of microalgae grown commercially include Chlorella, Spirulina, Dunaliella salina, Haematococcus pluvialis, Scenedesmus, Phormidium, Botryococcus, Chlamydomonas, etc.

The current global production of microalgae is about 20,000 dry tonnes per year. Two strains, Chlorella and Spirulina account for 90% of global production. These are processed for human food, extraction of nutraceuticals and animal food. A typical production rate for algae grown in suspension in water is in the range of 5-15 grams per day per meter square of reactor exposed to light (g/m²/d). CO₂ can be transferred from the air, but higher production rates are achieved by bubbling the water with a CO₂-rich gas. For these applications, commercial fertilizers are added as a source of nutrients.

There has been a large effort to grow algae for their energy content through conversion into biofuel. While research and development continue, production costs are still too high; a production target of 25 g/d/m² has been proposed to make algae cost effective to produce biofuels. Current efforts are focused on the production of sustainable aviation fuels.

More recently, there has been interest in growing algae to capture atmospheric CO₂ from the air to fight climate change. Useful products could be extracted from the algae before the residual carbon biomass is put away to sequester carbon.

Wastewater treatment is an emerging algae application. In addition to removing nitrogen and phosphorus that they need for growth, algae produce photosynthetic oxygen that can be used by bacteria in mixotrophic cultures to remove organic carbon (i.e., biological oxygen demand). Synergistically, the carbon dioxide produced by bacteria can be used as a source for algae growth.

The vast majority of commercial microalgal biomass is produced in ponds using natural light. Raceway ponds are shallow recirculating reactors where algae grow in suspension. The maximum concentration of suspended solids is circa 500 mg/L, limited by light penetration.

Raceway ponds occupy large areas of land. There have been efforts to intensify production by removing the rate limiting steps in the production of algae, such as algae concentration, carbon dioxide and nutrient concentration, surface area exposed to light, etc.

Algae process light through photosynthesis up to a flux of about 200 μmol photons/m²/s (26 kWh/m²/month). Above that, light saturation can be detrimental to growth. For example, in sunny climates such as the South of Spain, light flux can exceed this threshold by a factor of 5 in January and up to 10 in July.

Photobioreactors can be enclosed or exposed to atmosphere. Enclosed photobioreactors are preferred for high value-added products when it is beneficial to protect the algae from external contamination, or when using a gas source concentrated in CO₂. For other applications, reactors exposed to atmosphere may be simpler as they allow direct gas exchange.

U.S. Pat. No. 8,101,080 describes an enclosed photobioreactor consisting in a series of recirculating horizontal plastic tubes called tubular fence, filled with an algae suspension. The effluent is filtered through a membrane and a portion of the algae is returned to the reactor. Algae concentration in the reactor can reach 800-1,000 mg/L.

DE 10 2010 008 093 A1 describes an enclosed photobioreactor comprised of transparent hanging bags. The bags are deployed in rows with sufficient distance between rows to allow natural light to reach the algae suspension inside the bags. Algae can be further concentrated using a membrane filter.

U.S. Pat. No. 10,829,398 describes a membrane photobioreactor with light emitting diodes (LED) tubes immersed in the algal suspension.

As an alternative to suspended growth, microalgae can also be grown in a biofilm.

A rotating disk called AlgaDisk was developed in the framework of a European Union project for the purpose of carbon dioxide capture and biomass production. The disks are partially immersed and covered to control the gas atmosphere. A carbon dioxide rich gas is bubbled in the reactor. Another rotating biofilm support called the Algaewheel is described in U.S. Pat. No. 7,850,848. It is specific to wastewater treatment and involves floating wheels mounted on a shaft and positioned over aerators to impart a rotating movement.

U.S. Pat. Nos. 9,932,549 and 10,125,341 describe an algal biofilm growing on travelling flexible material mounted over rollers to circulate the algal population alternatively between an aqueous and a gaseous phase. The algae are harvested by scraping the biofilm off the travelling band.

EP 3360954A1 discloses growing algae attached to a floating fabric. Using a hydrophilic material for the fabric allows homogeneous and continuous wetting of the fabric.

U.S. Pat. Nos. 4,333,263, 4,496,096, 5,097,795, 5,572,770, 5,851,398, 8,375,627 and US20150189833 describe a system called the algal turf scrubber in which water flows over an inclined surface covered by a 3-dimensional screen that serves for benthic algae attachment. Algae are harvested by hydraulic or mechanical means.

US20170127656 describes vertical surfaces in the form of plates or cylinders hung over a water surface to support algae biofilm. A similar approach is disclosed in WO 2015131830A1.

### SUMMARY

This specification describes a system or device that can be used to grow algae substantially in a biofilm attached to a carrier. In some examples, the carrier is stationary.

The carrier has one or more vertically extending surfaces. Optionally, the vertically extending surface is inclined from the vertical. The surfaces may be planar or curved in one or two dimensions. The average inclination, measured on a straight line between the top and bottom of a surface, may be at a small angle, preferably between 2°-10°, most preferably between 3°-7°, to vertical. The one or more surfaces are vertically extending in the sense that their extension vertically is multiple (i.e. 3-50 or 8-20) times greater than their extension in at least one horizontal direction.

The surface may be configured to occupy a two-dimensional space or three-dimensional space. In the case of one or more surfaces occupying a two-dimensional space, carrier may be in the form of many discrete units, each having one or two surfaces, for example a V or U or an inverted V or U., or the carrier may be in the form of a continuous (or at least long) sheet with many (i.e. 3-1000) surfaces, for example a wave or an accordion. In the case of one or more surfaces occupying a three-dimensional space, the one or more surfaces may define for example a cylinder, cone, prism or pyramid. The surfaces may include a bottom of the three-dimensional space or the bottom may be omitted. The surfaces may include a top of the three-dimensional shape or the top may be omitted. Optionally, an upper part of a three-dimensional space may be truncated, for example an apex-truncated cone or pyramid. Two or three-dimensional spaces may be right (i.e., symmetrical about a vertical axis or one or more vertical planes) or oblique. In preferred examples, the carrier is in the form of an open-bottomed, apex-truncated, cone or pyramid.

An inclination to the vertical can also be expressed as an aspect ratio, which is defined as the ratio of height over a characteristic distance at the bottom of the carrier (for example, width or spacing between subsequent surfaces for an inverted V, base diameter for a cone, or base width for a pyramid). The preferred range of angles to the vertical corresponds to aspect ratios of about 10/1 (3°) and 4/1 (7°).

The carriers of the invention can be deployed on or over a reactor surface that is substantially horizontal to develop a larger surface area for growing algae per unit footprint. The ratio of carrier surface area exposed to light over footprint, called specific surface area, is a useful metric to express the intensification factor of the invention; this parameter is also often called the light dilution factor. As will be described in detail below, in some examples carriers of the invention can be deployed to develop a specific surface area of 10 m²/m² or more, optionally up to 25 m²/m².

The specific surface area is a function of the angle to the vertical (or the aspect ratio) and remains the same for any carrier size with the same aspect ratio. However practical considerations help define a range of preferred sizes. At the low end, the size should not be so small that 1) the specific surface area is lost when the carriers are covered with a thick layer of biofilm (i.e., there is bridging of adjacent surfaces by the biofilm), and 2) a space left between carriers at the base to allow water evacuation significantly reduces packing density. These considerations define a preferred minimum height of 0.1 m. At the high end, the size should not be so large that 1) the structure to maintain the carriers in their vertical position becomes complex and costly (given the weight from algal load and possibly wind forces), and 2) the energy required to irrigate the algae is prohibitive (as water may need to be pumped to the top of the carriers). These considerations define a preferred maximum height of 2 m.

So, the preferred carrier height range is from 0.1-2.0 m, most preferably 0.2-1.5 m.

The preferred carrier height corresponds to horizontal distances between crests for (for U, V or inverted V) or summits (for cones) that would allow circulation of operators on their top surfaces with or without light machines on wheels for the purpose of carrier installation, maintenance, irrigation or harvesting of algae. Operators may have to wear special shoes (like snow shoes) and light machines may have fat tires to spread their weight over multiple carriers.

The surface of the carrier is not limited to being flat (e.g. V, inverted V) or curved (e.g. cone) but could be pleated, undulating ore otherwise embossed to create local valleys and peaks and thus further increase the specific surface area. Optionally, the surface of the carrier may be formed of a flexible material, for example a fabric.

The carrier of the invention can be composed of one or more layers. The first layer may be solid but preferably, the 1^{st} layer is woven or nonwoven fabric, permeable to water and capable of filtering out algae at least to some extent, optionally after forming a dynamic filtration layer on the fabric. The first layer may be made of a hydrophilic material to improve water distribution over the carrier surface and delivery of nutrients to the algae. Preferably, a 2^{nd} layer is a mesh or screen that create alveoli where algae can grow protected from shearing by flowing water. Third or more layers may also be a mesh or screen. The layers are preferably directly adjacent to each other. Optionally, the layers are spaced apart from each other, for example by 0.1 to 1.0 mm or 0.5 to 10 mm, to provide a volume for biofilm growth between layers. Spacing between layers may be provided, for example, by undulations in one or more layers, by spacers, by variations in rigidity, tension or compression, or by variations in diameter.

The biofilm is kept wet, for example by irrigation, continuously or intermittently. In some examples, irrigation is 0.5-5 min on and 2-10 minutes off or 5-15 minutes off, for example 1 min on and 9 min off. Preferably, irrigation involves adding water near the top of the carrier and letting the water trickle down the surface. Alternatively, water may be applied, for example by a spray or drip, to some or all the surface of the carrier. Optionally, the biofilm may be kept wet by adding water to the outside of the carrier, for example by adding water directly to an outer surface of the carrier or to an algae film on the outside of the carrier.

The surface of the carrier may contain undulations or ridges whose purpose is to divert and redistribute the irrigation flow and/or to prevent channelling.

The carriers can be deployed over a reactor that contains water, but preferably they are deployed over a gently sloped surface where the irrigation water can be collected and recycled. Carriers can be self-standing, or preferably mounted on a structure where they are hanging. A carrier mounting structure can also include a hydraulic network used to convey irrigation water. Alternatively, the biofilm may be wetted by temporarily submerging the carriers, for example by dipping the carriers into water below the carriers or by temporarily raising the surface of water above the carriers.

In a reactor used to grow algae, the carrier's surface is exposed to light and in contact with a source of gaseous CO₂. The light is preferably natural, and the source of CO₂ is preferably air. The reactor has means to add feed water and remove effluent water. Feed water can be make-up water containing nutrients and effluent water can be water depleted in nutrients. Feed water also can be wastewater and effluent water can be treated wastewater. The reactor has means to recirculate water at a rate that allows keeping the algae wet and fed with nutrients, without abrading the algae from the biofilm.

Algae are periodically harvested from the carriers. For example, algae can be harvested by spraying the biofilm surface to detach the algae. The algae are collected after they detach from the carrier surface.

In some examples, the carriers may offer one or more benefits. First, the carriers may have a large specific surface area. For example, a V, an inverted V or a cone with an aspect ratio of 8 (angle to vertical) 3.6° has a surface area for growth over footprint of approximately 16/1. This specific surface area represents a potential intensification factor (ratio of algae production per unit footprint relative to that of a raceway pond) as the rate of algae growth is typically proportional to surface area, limited by light or CO₂ absorption from the ambient gas. Second, an angled surface area is easy to access from above for irrigation of the biofilm. Third, an angled surface area, when preferentially combined with a water permeable carrier fabric speeds up biofilm establishment and growth by having a portion of the irrigation water flow through the carrier. An angled surface may also contribute to suspended solids removal when used for wastewater treatment. Fourth, when using natural light, an angled surface might promote better light exposure to an array of carriers as the sun moves through the sky.

The algae carriers may be used to grow microalgae for one or more purposes, including 1) as food for human consumption or for the extraction of nutraceuticals, 2) as feed for aquaculture and animals, 3) for their fertilizer value, 4) for their energy contents to be converted into biogas or liquid fuel, 5) to biologically capture carbon dioxide for the purpose of sequestration, and 6) for wastewater treatment.

The reactor can be used to grow algae for their intrinsic value, optionally using the minimum amount of feed water containing nutrients, or it can be used to treat wastewater optionally while collecting algae as a valuable by-product and/or for carbon sequestration. The reactor design and its modes of operation are similar for both applications.

The shape of the reactor is not critical, but rectangular reactors are preferred. The reactor can be of any size, preferably in the range from 200-10,000 m², most preferably from 500-5000 m². For a large plant, multiple reactors can be used.

Overall, it is desirable that the reactor have minimum permanent supports and/or equipment overhead that could provide shade to the carriers.

The reactor may include a process tank or be connected to a process tank. When growing algae, a fresh nutrient solution is added to the process tank, and an equivalent amount of bleed water is taken out of the process tank, minus evaporation. When treating wastewater, flow through the process tank may be a function of the treatment capacity of the reactor for specific pollutants (e.g., nitrogen, phosphorus, BOD/COD). Water may be pumped from the process pump to an irrigation system continuously or intermittently to provide nutrients to the algae biofilm and to keep the algae biofilm wet.

A spraying harvesting network is also attached to the overhead distribution system.

Ambient or modified air may be used to provide gas transfer into the biofilm, CO₂ for algae growth and optionally O₂ for bacterial respiration (for example when treating wastewater with a mixotrophic biofilm at night). The reactor may define a path for air flow through the carriers which can be by natural convection or forced flow. Spray irrigation can also provide an opportunity for transfer of CO₂ and O₂ from/to the air Optionally, the reactor can be covered or enclosed, for example by a

greenhouse, to improve year-round operation at high latitude. In this case, it may be desirable to minimize the overall height of the reactor. When enclosed, the air inside can be enriched with a source of CO₂ to boost algae production

Artificial lighting can be added to the reactor to support photosynthesis during times of low or no solar irradiation (i.e. night, winter, dawn/dusk).

### BRIEF DESCRIPTION OF THE FIGURES

FIG. 1 shows the cross section of examples of carriers that are symmetrical to a vertical plane (U, V or inverted V) or to a vertical axis (cone).

FIG. 2 is a plan view a 1.0 m² section of reactor covered with inverted V (FIG. 2a) or cones (FIG. 2b).

FIG. 3 a graph illustrating the surface area ratio of a pleated fabric and a cone.

FIG. 4a and FIG. 4b show examples of reactors with carriers in the shape of a cone.

FIG. 5 is another example of a reactor with carriers in the shape of a cone.

FIG. 6a shows a reactor for growing algae in suspension in elevation view and

FIG. 6b shows part of the reactor of FIG. 6a in plan view.

FIG. 7 shows a reactor for growing algae on a stationary carrier in the shape of a cone.

FIG. 8a shows a reactor for growing algae on suspended carriers in the shape of cones. FIG. 8b shows a carrier of FIG. 8a.

FIG. 9 is a graph showing the correlation between algae concentration expressed as turbidity and total suspended solids.

FIG. 10 is a microscope photo of algae from an algae biofilm grown in an experimental example.

FIG. 11 shows the results of algae grown in suspension.

FIG. 12 shows the conditions for inoculation of a carrier.

FIG. 13 are pictures of carriers before and after harvesting algae.

FIG. 14 shows the results of algae grown on suspended cones

### DETAILED DESCRIPTION

The words “preferably”, “preferred” and similar words are used in this specification to refer to features that are optional and may be advantageous in at least some circumstances. The word “may” is also used to refer to something optional.

### Carriers

The description below uses examples of carriers that are either an inverted V or a cone. In these examples, the surface is inclined to the vertical and symmetric around a vertical plane or a vertical axis. Carriers in other two-dimensional or preferably three-dimension shapes, having other forms vertically extending surfaces, may also be used.

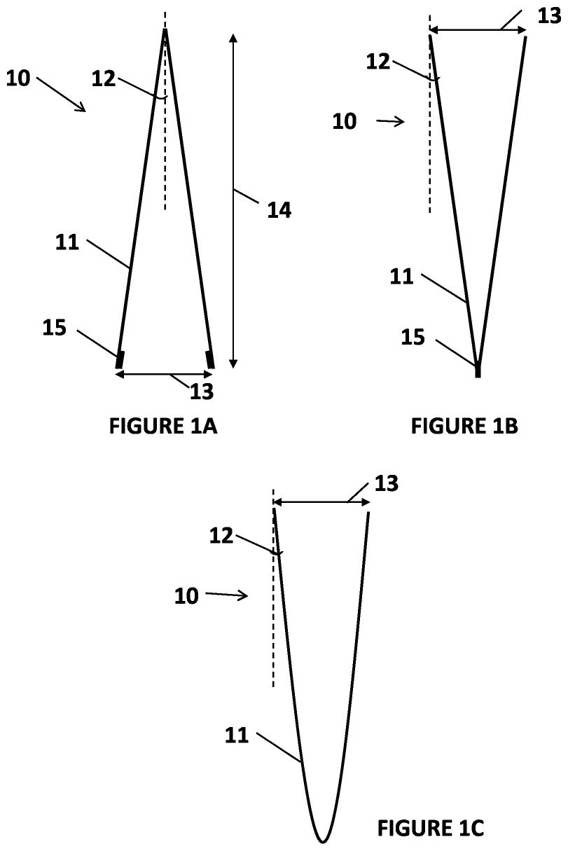

FIG. 1 represents 3 examples of the cross-section of the general configuration of a stationary carrier 10. FIG. 1a) is an example of an inverted V or a cone or pyramid; FIG. 1b is an example of a V shape or inverted cone or pyramid; and FIG. 1c is an example of a parabolic U-shaped carrier or an inverted cone or pyramid, all with the same preferred angle to the vertical and aspect ratio.

Angle 12 is defined as the inverse tangent of ratio of half of the width 13 divided by the height 14, independent of whether the actual carrier surface is a straight or not.

Angle 12 is preferably between 2°-10°, most preferably between 3°-7°. Angle 12 defines an aspect ratio which is equal to height 14 divided by width or diameter 13. The preferred range of angles from the vertical corresponds to aspect ratios preferably between about 14:1 and 3:1, most preferably between 10:1 and 4:1. Carrier 10 can be self-standing if its surface has a solid layer capable of maintaining its shape or if its surface is mounted on a frame. For this case the carrier could be sitting on the reactor surface, supported from below. Preferably, carrier 10 is suspended from its top portion, supported by one or several posts (not shown). Alternatively, carrier 10 could be hung from a cable or a structure above, as long as that structure does not create significant shade on the carrier surface.

Carriers supported from their top portion can be made from a flexible material if, for example, they have a relatively rigid or heavy structure at bottom 15 to maintain their shape. Carriers designed this way could be self-levelling. In the example of FIG. 1c, the U-shaped carrier is supported from its top portion and the flexible fabric is loosely hanging; its actual shape can be a function of its weight which may change with biofilm thickness.

As it relates to carriers symmetrical to a vertical plane, it will be understood that the examples in FIG. 1 represent a single fold of a continuous carrier that would be similar to an accordion or a pleated fabric.

Carrier 10 can have a section of its top portion cut-off or truncated, typically 5-20%, to create a space sufficient to contain a support mechanism and potentially a water distribution means for irrigation.

The nature of the carrier surface is not critical as it is well known that algae can attach to almost any surface given enough time. However, it is preferred that the carrier has one layer made of a fabric with the following characteristics: non-biodegradable, strong, UV resistant, porous, and hydrophilic. Strength is required to resist the algal load and any applied forces (e.g., wind, water sprays). UV resistance is preferable for durability. Porosity and hydrophilicity are preferable to allow water passage during start-up to accelerate biofilm establishment and to transport nutrients. Porosity should preferentially be in a range between 25-75%, with pore sizes 2-100 μm or 2-20 μm. Hydrophilicity can be measured by water absorption capacity, which may be at least 20g/100 g of material, as measured for example by method ISO2417. Preferred materials for the first layer include woven or nonwoven fabrics from material including, but not limited to polyester, polyethylene, polypropylene, nylon, etc. The material for the first layer could also be chosen among low carbon footprint materials such as cotton, hemp, jute, linen/flax, sisal, bamboo, coir, grasses, etc. The material for the first layer may be rigid or flexible.

An optional second layer could consist of a mesh or netting material capable of creating alveoli to retain algae in the biofilm and prevent premature slough off. Preferred materials for the second layer may be similar to the first layer but with grid openings of 1-10 mm. Third or more layers may be similar to the second layer.

An alternative to a second layer consists of embossing, pleating or otherwise shaping the first layer to create the desirable alveoli.

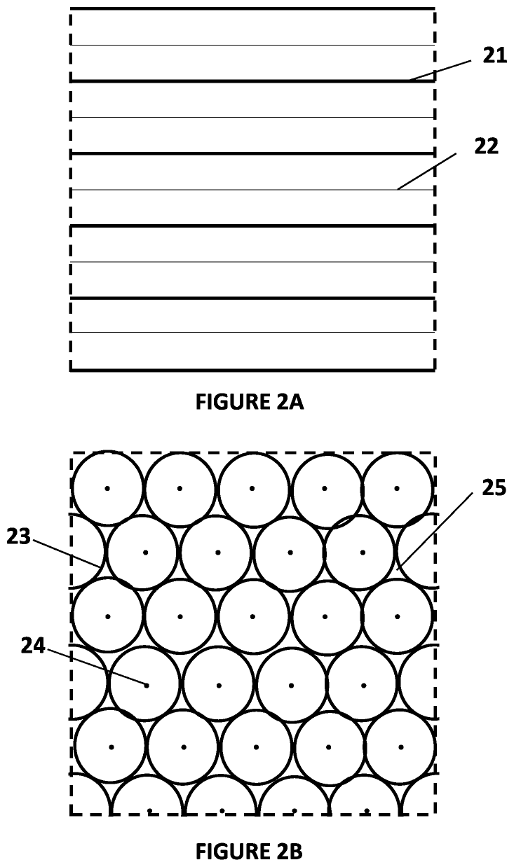

FIG. 2 shows how inverted Vs and cones can be deployed to cover 1 m² of the surface of a reactor. These were drawn to scale to represent carriers with an aspect ratio of 8 (angle from vertical of) 3.6° having a height of 160 cm and a base of 20 cm. In FIG. 2a, the bases of inverted Vs are represented by thick lines 21 and the crests by thin lines 22. There are 5 carriers per m² of reactor surface area, each measuring 1 m long. This accordion pattern has a surface area for biofilm growth of 16 m², or a specific surface area of 16 m²/m². FIG. 2b shows cones in hexagonal packing (rows offset by half a diameter). There are 28.9 cones per m² with a specific surface area of 15.4 m²/m².

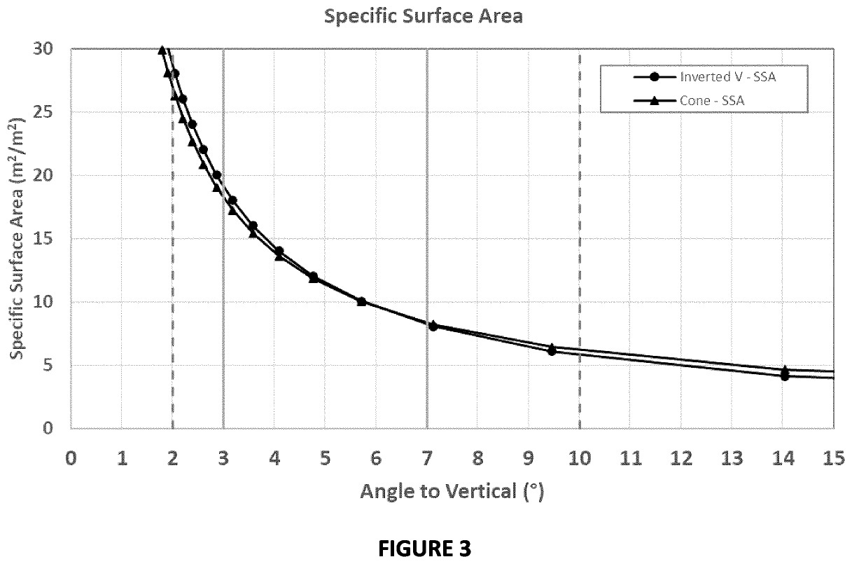

FIG. 3 is a graph showing the theoretical maximum specific surface area as a function of the angle to vertical for inverted Vs and cones packed as illustrated in FIG. 2. The range of preferred angles (2°-10°) is within the vertical dotted lines while the most preferred angles (3°-7°) is within the vertical solid lines. This graph shows that the inverted V has a slightly higher theoretical maximum specific surface area as compared to the cone within the most preferred range.

The actual specific surface area deployed in a reactor is typically lower than the theoretical maximum surface area represented by FIGS. 2 and 3. For example, a portion at the top of the inverted V and the cone may have to be sectioned or truncated to position supporting brackets and/or irrigation means. At the bottom, a gap may be needed between inverted Vs for flow of irrigation and harvesting water to the reactor floor below. For cones, hexagonal packing may leave a sufficient flow gap provided that the cones are within the preferred size range of 0.1-2 m, but spacing can be increased to achieve any gap size.

The table below is an example of theoretical maximum and actual specific surface areas for carriers within the most preferred range, with a height of 160 cm and base of 20 cm. A 15% section is cut off at the top for both the inverted V and the cone, leaving a space of 3 cm for attachment to a supporting structure and/or irrigation means. At the bottom, there is a gap of 2 cm between inverted Vs, but there is no need for an additional gap between rows of cones as there already are openings of about 3 cm between cones as illustrated in FIG. 2b. It should be noted that the carrier projected surface area of 0.907 represents the maximum packing density for circles in a hexagonal pattern. The actual specific surface area is reduced to 12.4 and 15.1 m²/m², respectively for the inverted Vs and the cones. This analysis shows that, for the same aspect ratio, the cone offers a 22% higher actual specific surface area.

The actual specific surface area can be used to extrapolate the surface production of a carrier to an aerial production.

| | Units | Inverted V | Cone |
|---|---|---|---|
| Aspect ratio | | 8 | 8 |
| Height | cm | 160 | 160 |
| Base | cm | 20 | 20 |
| Theoretical Maximum Specific Surface | m²/m² | 16.0 | 15.4 |
| Area | | | |
| Portion cut-off at the top | | 15% | 15% |
| Dimension where cut-off | cm | 3.0 | 3.0 |
| Net height | cm | 136 | 136 |
| Bottom gap | cm | 2.0 | 0.0 |
| Carrier project surface area coverage | fraction | 0.907 | 0.907 |
| Actual Specific Surface Area | m²/m² | 12.4 | 15.1 |

### Reactors

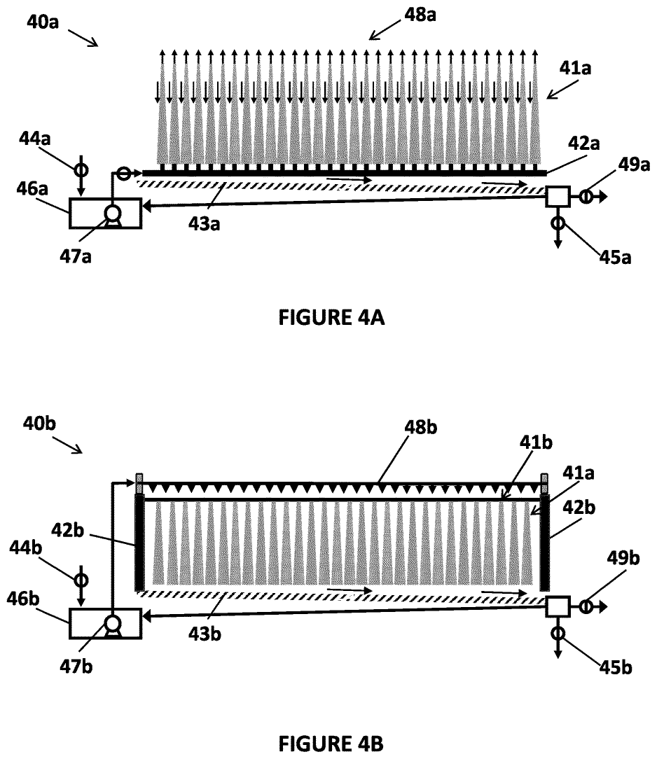

FIG. 4a is an example of a reactor 40a where the carriers 41a are mounted on a hydraulic network 42a. The hydraulic network is substantially leveled but is mounted on a reactor floor 43a which is sloped to recover irrigation water. Reactor 40a has means to add feed or make-up water 44a containing nutrients and to withdraw effluent 45a depleted in nutrients. Feed 44a is added to a tank 46a which contains irrigation water, from which a pump 47a can distribute it to the carriers through hydraulic network 42a. Each carrier 41a is mounted on a standpipe having a distribution cap 48a which allows distribution of irrigation water flowing upwards from hydraulic network 42a through the standpipes and the distribution caps 48a to the outside of the carriers 41a near the top of the carriers 41a. From there, irrigation water trickles down the outside of the carriers and is collected on sloped floor 43a from where it flows back to tank 46a.

FIG. 4b is an example of a reactor 40b where carriers 41a are suspended from cables 41b or other suspending structures such as beams, which may be arranged in parallel or in a grid. The cables 41b are attached to support structures 42b located on opposite sides of the reactor 40b. The carriers 41a are self orienting (i.e. they hang generally vertically) and are mounted at substantially the same elevation (with some variation for the cable sag). The reactor floor 43b is sloped to recover irrigation water. Irrigation water is distributed to the top of the carriers from above through an irrigation system, in the example shown having nozzles mounted on a moving bridge 48b. In this embodiment, bridge 48b travels on top of support structure 42b, but alternative mounting arrangements are possible. In the example shown, the bridge 48b moves at right angle to the direction of the suspension cables (i.e., in and out of the page). As the moving bridge 48b travels across the bed of the reactor 40b, individual carriers 41a are sprayed with water intermittently but the water is dispersed over the bed area (i.e. floor 43b) of the reactor 40b. Other hydraulic functions of reactor 40b are like reactor 40a and will not be repeated here for brevity.

In the embodiments represented by FIG. 4a and FIG. 4b, inorganic carbon required for algae growth is mostly transferred directly to the biofilm from ambient gas, but it could also be dissolved into tank 46a,b. The reactor 40a,b may behave like a completely stirred tank reactor (CSTR) if the recirculation of irrigation water is sufficiently greater than the feed/effluent flow. In some cases, especially involving wastewater treatment, it may be desirable to deploy multiple reactors like reactor 40a,b in series to approach a plug flow mode of treatment.

In the mode of operation described above, algae slough off from the carriers when the biofilm becomes too thick, and they are recovered with the effluent. As an alternative, reactor 40a, b can be turned into harvesting mode by stopping feed 44a,b and diverting effluent 45a,b to a harvested algae stream 49a,b. In this mode, the carriers may be sprayed to remove algae from the surface of the carriers and to recover algae in a concentrated form. The carriers 41a and algae films attached to the carriers 41a are wetted by irrigation water applied to the outside of the carriers 41a. In these examples, irrigation water does not flow outwards through the algae supporting surfaces of the carriers 41a to wet the algae films.

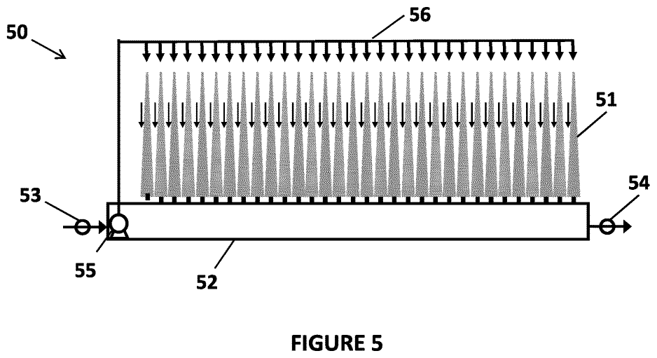

FIG. 5 is an example of a reactor 50 where the carriers 51 are mounted on top of a tank 52. They can be mounted on posts (as shown) or suspended from a suspending structure (similar to FIG. 4b, not shown). Reactor 50 has means to add feed or make-up water 53 containing nutrients and to withdraw effluent 54 depleted in nutrients. Feed 53 is added to tank 52 which contains irrigation water, from which a pump 55 can distribute it to the carriers through a fixed hydraulic network 56 with nozzles directed at the tops of carriers 51 (as shown) or with fixed wider angle spray heads to disperse water across the bed area of the reactor 50, or through a moving bridge 48b similar to FIG. 4b (not shown). Irrigation water trickles down the carriers and flows back to tank 52. One or more features of reactor 50 may be substituted or combined with one or more features of reactor 40a or reactor 40b.

Alternatively, carriers may be irrigated by temporarily immersing the carriers in water. For example, in a modified reactor 40b, the carriers 41b may be suspended over a tank 52 as in FIG. 5 (but preferably a deeper tank) in place of the reactor floor 43b. One end of the cables 41b may be attached to a bar that is pulled tight or relaxed by a hydraulic cylinder attached to one of the support structures 42b. When tension in the cables 41b is released, the carriers 41a are lowered into the water in the tank 52. Tightening the cables 41b lifts the carriers 41a back out of the water. The action of dipping the carriers 41a may release at least some of the algae from the carriers 41a but additionally or alternatively algae may be harvested from the carriers 41a by spraying water at the carriers 41a. The carriers 41b may also be intermittently lowered into water for other purposes, for example to protect the carriers 41b or algae from inclement weather if the carriers 41b are deployed outdoors. In another option, a tank 52 may have sides extending above carriers 41a or 51. The water level in the tank 52 may be raised and lowered to intermittently immerse the carriers 41a or 51.

### Experimental Methods

Validation experiments were done with a mixed culture of Chlorella vulgaris obtained from the Canadian Phycology Culture Centre (Waterloo, Ontario) and Tribonema minus obtained from Cal Poly (San Luis Obispo, California). Micro photos of the algae were taken with a AmScope M170C-E 40X-1000X Dual LED Portable Compound Microscope with Camera.

The algae were grown using Miracle GRO 24-8-16 at a concentration of 200 mg/L as a source of nutrients and, soda (NaHCO₃) at a concentration of 200 mg/L as an additional source of inorganic carbon and to increase pH and promote better uptake of atmospheric carbon dioxide. These chemicals were dissolved in de-chlorinated tap water from the City of Hamilton, Ontario.

Experiments were conducted in different reactors.

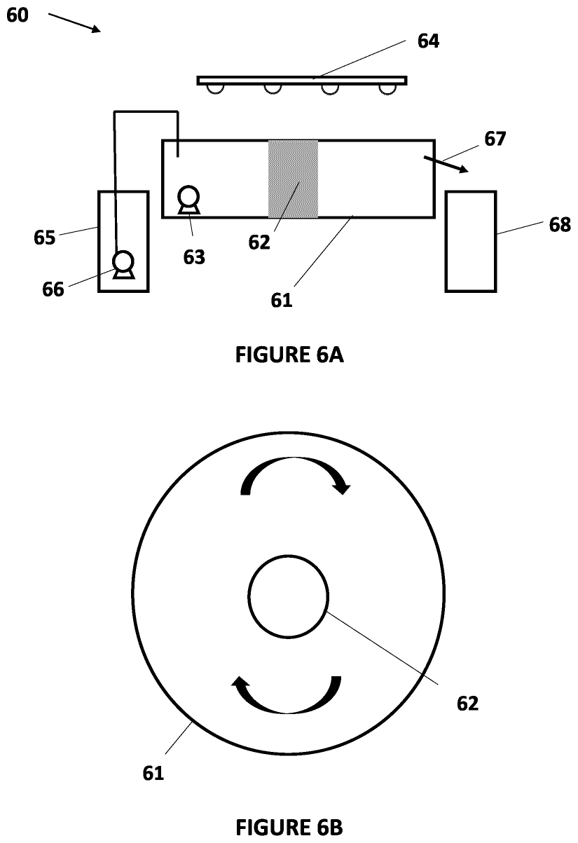

The reactor shown in FIG. 6 was dedicated to growing algae in suspension as a control. FIG. 6a shows a cross section; FIG. 6b is a plan view of the tank and centre isle showing the flow plan. Reactor 60 consisted of a 50 L tank 61 with a center isle 62 and a net surface area of 0.47 m². A submerged pump 63 was used to create a circulation movement in the tank. The pump, Coral Box model QPS9, created a velocity of 20-30 cm/s, sufficient to prevent settling of suspended algae, and could also be used in a pulsing mode. Eight LED lights 64 from FEIT model GLPFS/19W were positioned above the tank to generate a photon intensity of 150 μmol/m²/s on a continuous basis (measured using a Light Scout 3415A meter). A system was put in place to pump a feed solution into the reactor on a semi-continuous basis. It consisted of a feed pail 65, a feed pump 66, an overflow tube 67 and an effluent pail 68. The programmable pump 66 from Moisten Land, model 201, was turned on regularly to generate a flow of 5-10 L/d, which corresponded to a hydraulic retention time of 5-10 days.

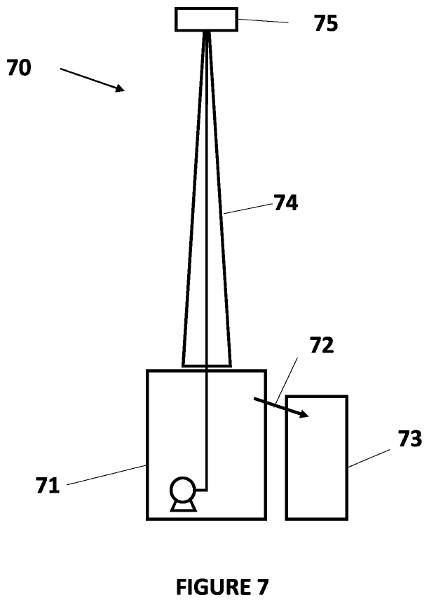

The reactor shown in FIG. 7 was used to grow attached algae with a conical carrier. The reactor consisted of a pail 71 with a working volume of 10 L and an overflow tube 72 into an effluent pail 73. A carrier in the form of cone 74 was positioned over the reactor, hanging from a bracket above the cone 74. Feed was added manually to the reactor, 2 L in the morning and 2 L at night. An irrigation water distribution box 75 was located above the cone 74 and had 16 holes of 1.6 mm diameter each equally spaced around its perimeter (on a 4 cm diameter). The holes of the irrigation water distribution box 75 were located outside of the perimeter of an adjacent part of the cone 74 such that irrigation water was delivered to the outside of the top of cone 74 and then trickled down the outside of the remainder of the cone 74. An aquarium pump (Cadrim rated 1200 L/h) submerged in pail 71 pumped irrigation water through a tube connected to distribution box 75. The instantaneous flow rate was set with a restriction valve to 30 L/h and was used intermittently, 1 min on, 5 min off. This generated a trickling flow along the surface of the cone. Eight LED lights (not shown) identical to those used for the suspended growth reactor were positioned around the cone to provide light at a photon intensity of 150 μmol/m²/s on a continuous basis.

The carrier was inoculated by filling the reactor with a suspension of algae taken from the effluent pail of the suspended growth reactor, diluted 1/1 with tap water. The pump was then started in recirculation loop for half a day before starting to add feed.

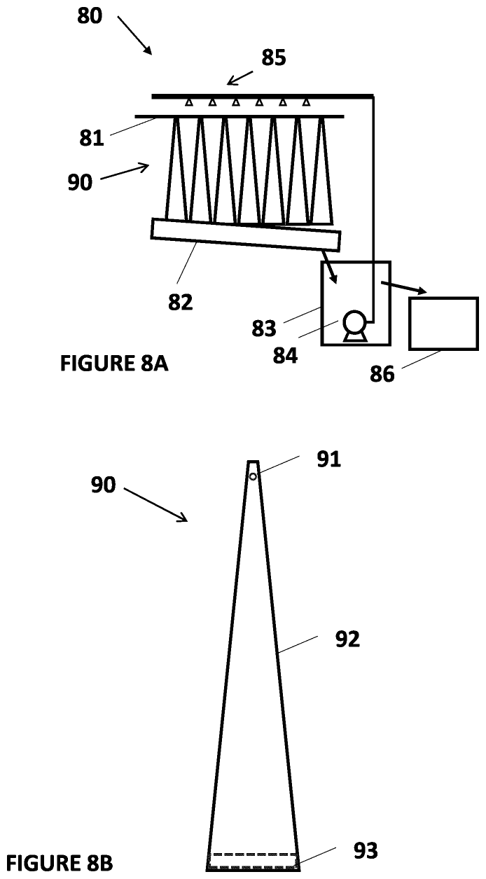

Reactor 80 shown in FIG. 8a was used to grow algae on carriers in the form of fabric cones 90 suspended on cable 81. The reactor consisted of tray 82 set with a slope and an opening to collect irrigation water and return it to pail 83. Pump 84 was used to recirculate water from pail 83 to irrigate the cones 90 through 6 stationary nozzles 85 located above the cones. Pump 84 was a small misting pump capable of generating a flow of 2.5 liters per hour through misting nozzles 85. The pump was controlled with a timer and was on for 30 seconds and off for 2 minutes. Pail 83 contained 10 liters and was fed with 2 liters per day of a fresh solution of nutrients as described above. It overflowed into effluent pail 86. Four LED lights 87 identical to those used for the suspended growth reactor were positioned on both sides of the cones 90 to provide light at a photon intensity of 100 μmol/m²/s on a continuous basis.

FIG. 8b shows further details of the cones 90. The surface of the cone is formed by flexble fabric 92. Each cone 90 had a fixture 91 for attaching the cone 90 to the cable 81. In the example shown, the fixture 91 is a grommet inserted through both sides of the fabric 92. In use, the cable 81 passes through the fixture 91. The bottom of the fabric 92 is kept open by a rigid frame 93. In the example shown, the frame 93 creates a circular opening. Cone 90 is described further in the experimental section below.

Water in the reactors was monitored daily for pH and temperature using a Hanna 98129 tester, and turbidity using a Hanna HI 3415A meter.

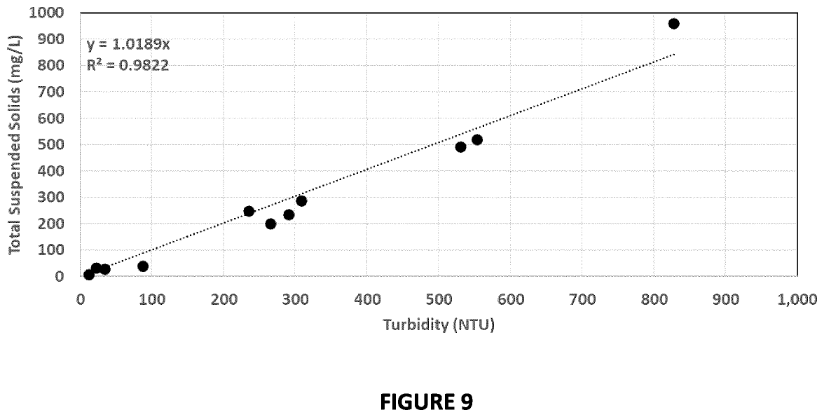

Turbidity was converted to suspended solids using the correlation shown in FIG. 9. Total suspended solids (TSS) were measured using a standard method by ActLabs Agriculture, Ancaster, Ontario. The correlation shows a roughly 1 to 1 relationship between turbidity and TSS, i.e., 1 NTU≈1 mg/L. It was assumed that a TSS measurement represent the dry mass of algae without any other correction.

### Experimental Carriers

Three cones of identical dimension were built and tested sequentially in the reactor shown in FIG. 7. They had an aspect ratio of 8 (angle to vertical of) 3.6° with a diameter at the base of 18 cm and an 18% section cut off at the top, which left a net height of 118 cm. They had a biofilm growth surface area of 0.42 m², a footprint of 0.028 m², corresponding to a specific surface area of 15.0 m²/m². The cones could be attached to the irrigation distribution box described above (75 in FIG. 7) and thus hang over reactor 70.

Cone 2511 was built with 3 layers. The first layer was a solid sheet of polystyrene, 0.76 mm thick, which was glued in the shape of a cone. This first impermeable layer was covered with a second layer of self-adhesive fiberglass tape from a local hardware store. The third layer was a material called Synlap, a synthetic burlap with a large mesh used to cover shrubs over the winter.

Cone 0212 did not have a solid base but was built with 3 layers of fabric obtained from a local store. The first layer was a tight weave of polyester fabric. The second layer was polyester tulle with openings of about 1 mm. The third layer was polyester poly-mesh with openings of about 5 mm. These fabrics were cut to a pattern, sewed together with stitch lines about 30 mm apart and then sewn into a cone. At the top, fabric was glued to a short piece of pipe for attachment to the reactor. At the bottom, belt loops were sewed on the inside to insert a plastic ring of 5 cm wide to expand the base into a circle. As a result of the stitch lines and differences in the material of the layers, wrapping the layers in a cone caused the layers to separate from each other to a varying degree, in some places by a few mm.

Cone 1012 was identical to Cone 0212 with the exception that the stitch lines were about 10 mm apart to limit separation of the layers of fabric from each other. A plastic ring was also added at mid-height to help redistribute irrigation water along the circumference of the cone and reduce channeling.

The hydrophilicity of the first and third layers of fabric used to make cones 0212 and 1012 was measured by the method described above. Both fabrics spontaneously absorbed water and had an absorption capacity of about 150 grams water per 100 grams of fabric.

Smaller cones were tested in the reactor described in FIG. 8a. Cone 1108 shown as cone 90 in FIG. 8b had an aspect ratio of 8, with a truncated length of 36 cm and a diameter at the base of 6.4 cm. It was sewed together from 2 layers of fabric, a woven polyester as base and an open mesh tulle as second layer. Seven (7) cones 1108 were hung side by side on a cable to provide total surface area of 0.35 m², a footprint surface area of 0.028 m², corresponding to a specific surface area of 12.4 m²/m².

### Experimental Result—Algae

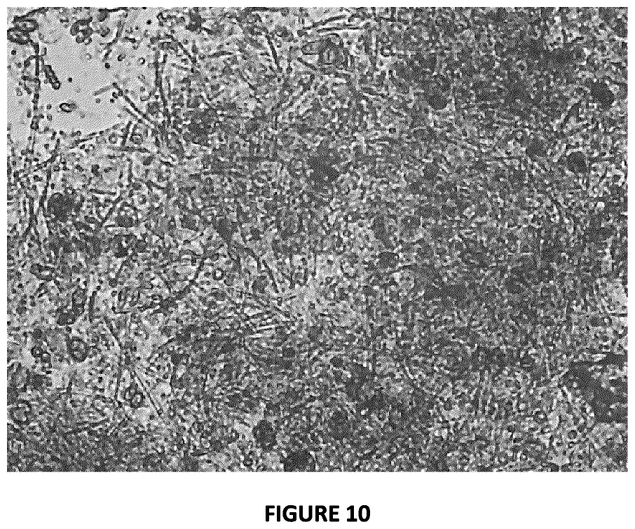

FIG. 10 is a microscope picture of algae from a biofilm showing Chlorella vulgaris (round shape) and Tribonema minus (filamentous shape). There is no visible bacterial growth or contamination such as would be present in wastewater.

### Experimental Result—Algae Grown in Suspension

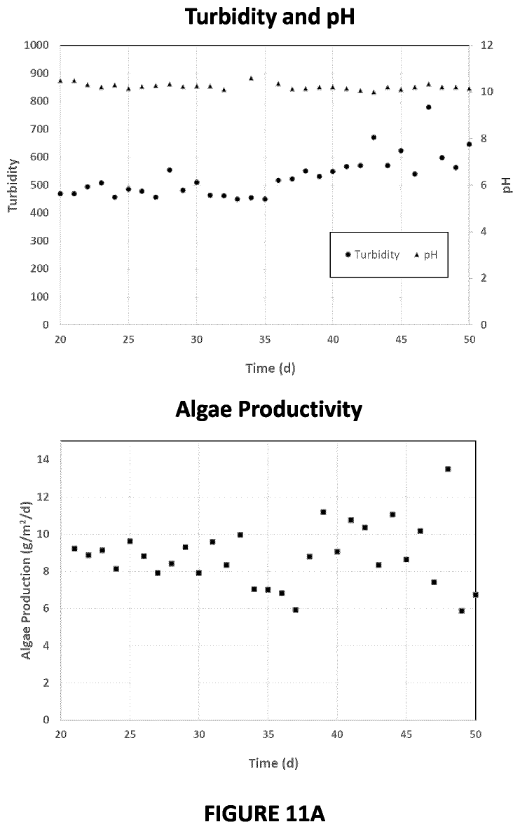

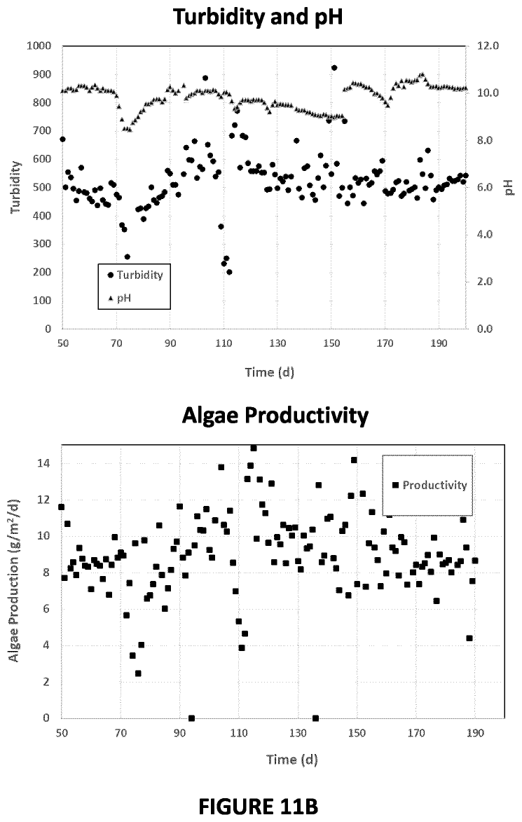

FIG. 11a shows the results of algae growing in suspension over a period of 30 days corresponding to the period when cones 2511, 0212 and 1012 were tested. pH was stable, just above 10.0 for the duration of the experiment. A high pH is an indication of a healthy algae population as the inorganic carbon in solution (from alkalinity and added sodium bicarbonate) has been mostly depleted. Turbidity ranged between 500-600 NTU. The temperature was stable, averaging 20.5° C. over the duration of the experiment. Turbidity was converted into algae production rate using the correlation presented in FIG. 9. Algae production averaged 8.6 g/d/m².

FIG. 11b shows the results of algae growing in suspension over a period of 150 days corresponding to the period when cones 1108 were tested. pH was more variable due to reactor upsets, but generally around 10.0 for the duration of the experiment. Turbidity and temperature were stable and similar to the previous experiment. Algae production averaged 9.0 g/d/m².

### Experimental Result—Algae Grown on Carriers

Each of the three cones 2511, 0212 and 1012 were tested for a period of 7-10 days. At the conclusion of an experiment, cones were sprayed using tap water and a high-pressure nozzle set to a flow rate of 1 L/min to remove the algae biofilm. The biofilm was collected in a suspension with the wash (sprayed) water. The algae suspension was blended to break up large particles and diluted to estimate the mass of algae from turbidity measurement.

The primary purpose was to determine the algal load (mass of algae per carrier) through a mass balance rather than simulating a harvesting operation. In a harvesting operation, it would be desirable to leave some algae on the carrier to speed up re-establishment of the biofilm when put back into operation.

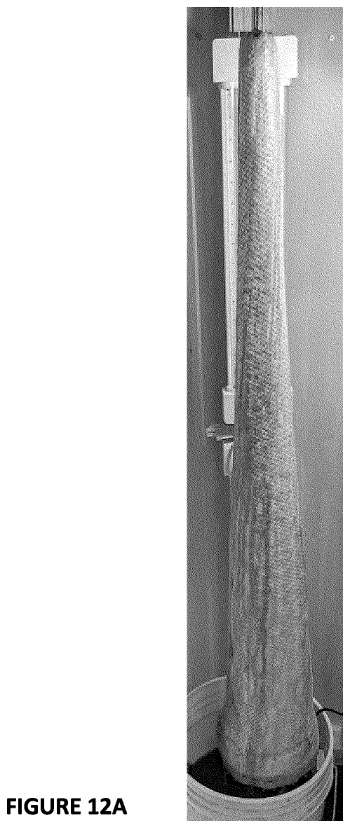

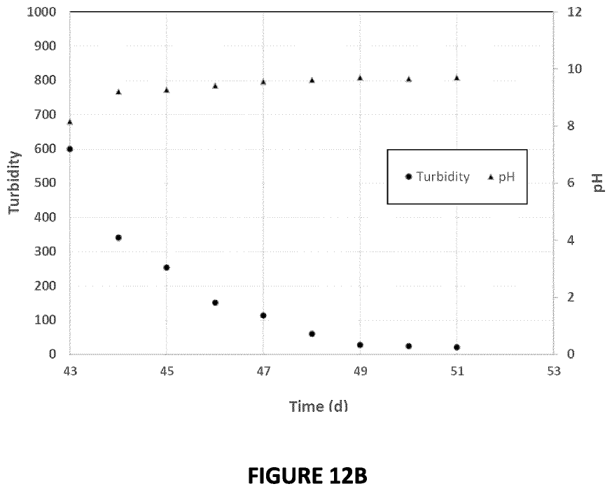

In order to better estimate the mass of algae that grew on the surface of the carriers, it is necessary to subtract the mass used for inoculation. As mentioned above, the cones were inoculated by filling the reactor (10 L) with an algae suspension. For example, FIG. 12a is a photo of cone 1012 taken 2 hours after inoculation that shows significant algae deposition on the fabric surface of the carrier (though not easily visible in a black and white picture). FIG. 12b is a graph showing that the initial turbidity of 600 NTU quickly dropped, confirming the visual observation of FIG. 12a. Turbidity continued to drop over the duration of the experiment, partly due to deposition, partly due to wash out with addition of fresh feed water (4 L/d). It was estimated that about 3 g of algae were deposited on the cone due to inoculation (half of the mass contained in the inoculation volume of 10 L @ 600 NTU). Similar estimations were done for cones 2511 and 0212.

Experimental results are summarized in the table below. In all three experiments, the algae were recovered at a relatively high concentration of about 5 g/L in wash water. This is about an order of magnitude higher than the concentration in the suspended growth reactor.

The experiment with cone 2511 was stopped after 7 days as significant channeling of irrigation water was observed; surface coverage was estimated visually to be about 40%. The net algae load of 8.1 g was equivalent to a surface production of 2.7 g/m²/d and an aerial production of 41 g/m²/d.

An increase in visual coverage to about 60% was observed with cone 0212. The increased coverage might be a result of eliminating the solid layer or using layers of more hydrophilic fabrics. Surface production increased to 4.4 g/m²/d and aerial production of 66 g/m²/d.

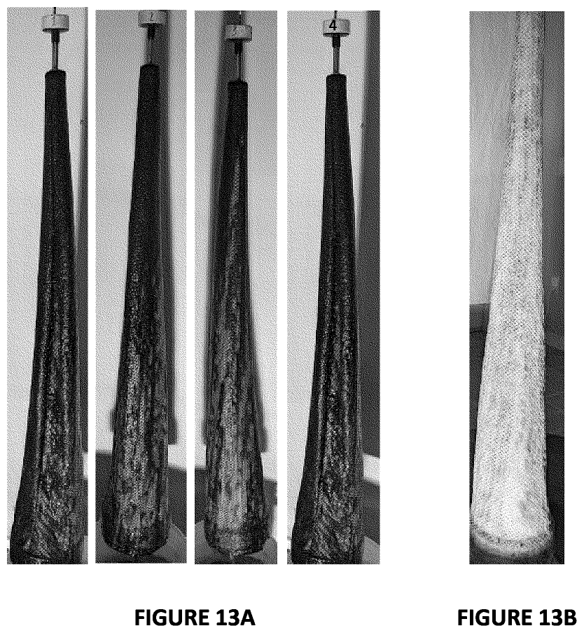

A further improvement was observed with cone 1012, with visual algae coverage increasing to about 80%. The increased coverage might be a result of stitching fabric layers closer together or redistribution of the irrigation water by the half-height ring. Cone 1012 is shown before and after algae harvesting by spraying in FIGS. 13a and 13b. FIG. 13a shows the cone before spraying from 4 different angles; FIG. 13b shows the cone after spraying. Surface production was 7.9 g/m²/d and aerial production of 119 g/m²/d. Surface production is almost the same as the control reactor (8.6 g/m²/d) and aerial production is 14 times larger than the control reactor, which validates the specific surface area concept introduced above as an intensification and scale-up factor.

These experiments involved single carriers exposed to uniform light intensity and did not simulate shade effects that would occur in a packed cone reactor. However, the light intensity of 150 μmol/m²/s would be representative of diluted light reaching the biofilm in a tightly packed cone reactor on a sunny day. While it is impossible to directly extrapolate our results to actual field conditions, the benefits relative to the control suspended growth reactor should hold true under most field conditions.

As indicated in the table below, the cones had an aerial production of over 40 g/m²/d. In comparison, the aerial production of the suspended growth reactor described above was 8.6 g/m²/d.

| Cone | 2511 | 0212 | 1012 |
|---|---|---|---|
| Experiment duration (d) | 7 | 10 | 8 |
| Visual coverage | 40% | 60% | 80% |
| Algae concentration in wash water (g/L) | 4.7 | 5.0 | 4.6 |
| Total algal load (g) | 9.1 | 20.4 | 29.6 |
| Estimated load from inoculation (g) | 1.0 | 2.0 | 3.0 |
| Net algal load (g) | 8.1 | 18.4 | 26.6 |
| Surface production (g/m²/d) | 2.7 | 4.4 | 7.9 |
| Areal production (g/m²/d) | 41 | 66 | 119 |

The turbidity and pH curves of FIG. 12b are worth additional analysis. The initial pH of the inoculation solution, 8.2, gradually increased to above 10, which an indication of a healthy algae population that consumed most of the inorganic carbon in solution. The initial turbidity of the suspension, 600 NTU, gradually decreased to almost zero; this is an indication that suspended solids were removed through contact with the carrier and that algae were not abraded from the biofilm through irrigation. The effluent might not require further treatment before discharge.

Cones 1108 were tested over a period of over 100 days. In this experiment, the reactor went through repeated cycles of growth and harvest without re-inoculation. Harvesting was done as described above for single cones.

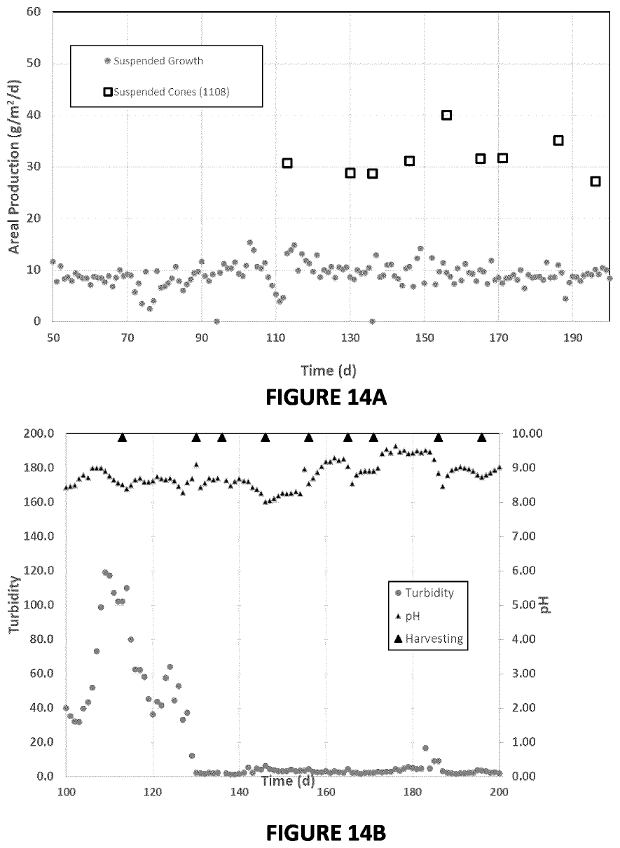

Results are shown in FIGS. 14a and 14b. FIG. 14a shows the areal production in comparison to the suspended growth reactor results. Over the observation period, cones 1108 were harvested 9 times at an average 10-day interval. The areal production averaged 31.7 g/m²/d or about 4 times higher than the production of the suspended growth reactor (9.0 g/m²/d). Effluent water quality is presented in FIG. 14b. Turbidity was high during a few weeks when the cones were sprayed daily with a suspension of algae (from the suspended growth reactor) for inoculation. However, after that initial period, turbidity dropped to below 10 NTU and was not affected by the harvesting events. pH ranged from 8-10; it tended to drop after a harvesting event, but quickly recovered within a few days.

When harvesting, the flow of spray water containing algae was collected into a separate pail (i.e., it was not allowed to flow into pail 83 shown in FIG. 8). The amount of water for harvesting was 8.1 L/m² of cones and the algae concentration was 2.3 g/L, on average. Prior to blending to measure turbidity and suspended solids, the harvested algae suspension was subjected to settling tests and compared to settling of an algae suspension removed from the suspended growth reactor. For the suspended growth reactor, no significant settling was observed over a period of 1 hour. This is attributed to the fact that algae grow as individual cells or small aggregates in suspension and are more or less buoyant. This is a well know characteristic of algae grown in raceway ponds. For the biofilm reactor, the algae quickly settled to about 10% of their original volume, leaving a relatively clear suspension above. This is attributed to the fact that algae grown in a biofilm detach as large blobs that easily settle. The settled algae concentration was about 23 g/L. The volume of water used for harvesting represented about 1.5% of the water treated through the reactor.

1. A carrier for growing algae having one or more surfaces, each with a vertically extending surface.

2. (canceled)

3. (canceled)

4. The carrier of claim 1 wherein the surface is inclined, on average or locally, between 2°-10°, optionally between 3°-7°, from the vertical.

5. (canceled)

6. (canceled)

7. The carrier of claim 1 having a surface surrounding a vertical, or vertically extending, axis.

8. The carrier of claim 7 having a surface symmetrical about a vertical axis, for example in the shape of a cone or a truncated cone or having a surface in the shape of a right pyramid or truncated right pyramid.

9. (canceled)

10. The carrier of claim 1 having a height in a range from 0.1-2.0 m or 0.2-1.5 m.

11. (canceled)

12. The carrier of claim 1 having a surface comprising two or more layers, optionally two or more layers of porous material.

13. The carrier of claim 1 having a layer of woven or nonwoven fabric, optionally permeable to water and capable of filtering out algae at least to some extent.

14. The carrier of claim 1 having one or more layers of a mesh or screen.

15. The carrier of claim 1 having two or more layers spaced apart from each other across most (i.e. 50% or more or 80% or more) of their surface area.

16. (canceled)

17. The carrier of claim 1 having a fixture near a first end of the carrier for hanging the carrier.

18. (canceled)

19. The carrier of claim 17 having a flexible surface and frame for holding a second end of the carrier open.

20. A reactor having one or more carriers of claim 1 deployed on or over a floor or bed area, optionally wherein the floor or bed area comprises a sloped surface or wherein the floor or bed area is part of a tank.

21. The reactor of claim 20 wherein the one or more carriers have a specific surface area of 7 m²/m² or more or 10 m²/m² or more, optionally up to 25 m²/m².

22. (canceled)

23. (canceled)

24. The reactor of claim 21 comprising an irrigation system having one or more nozzles adapted to spray water near the top of the one or more carriers or to disperse water across the floor or bed area of the reactor.

25. (canceled)

26. The reactor of claim 20 wherein the one or more carriers are supported on a surface such as a floor or bed area or suspended over the surface, for example on a suspending structure such as a cable, beam or grid.

27. The reactor of claim 20 wherein the irrigation system is adapted to recirculate irrigation water, for example at a rate that allows keeping the algae wet and fed with nutrients, without abrading the algae from the biofilm.

28. (canceled)

29. (canceled)

30. (canceled)

31. A process for growing algae comprising,
providing a carrier according to claim 1,
exposing the carrier to light, and
wetting the carrier.

32. (canceled)

33. (canceled)

34. The process of claim 31 wherein the carrier is suspended from a first end of the carrier.

35. (canceled)

36. The process of claim 31 wherein the carrier is wetted by spraying the outer surfaces of the carriers through multiple irrigation nozzles.

37. The process of claim 36 wherein irrigation water is recirculated to the carrier, for example at a rate that allows keeping the algae wet and fed with nutrients, optionally without abrading the algae from the biofilm.

38. (canceled)

39. (canceled)

40. (canceled)

41. (canceled)
